(19) United States
(12) Patent Application Publication
(10) Pub. No.: US 2024/0150724 A1
(43) Pub. Date: May 9, 2024

(54) PRODUCTION OF HEME FOR CELL-BASED MEAT PRODUCTS

(71) Applicant: Terasaki Institute for Biomedical Innovation, Los Angeles, CA (US)

(72) Inventors: Seyedvahid Hosseini, Newbury Park, CA (US); Alireza Khademhosseini, Los Angeles, CA (US)

(73) Assignee: Terasaki Institute for Biomedical Innovation, Los Angeles, CA (US)

(21) Appl. No.: 18/281,032

(22) PCT Filed: Mar. 9, 2022
(86) PCT No.: PCT/US2022/019615

§ 371 Date: Sep. 8, 2023

Related U.S. Application Data
(60) Provisional application No. 63/159,403, filed on Mar. 10, 2021.
(60) Provisional application No. 63/164,387, filed on Mar. 22, 2021.
(60) Provisional application No. 63/164,397, filed on Mar. 22, 2021.
(60) Provisional application No. 63/279,642, filed on Nov. 15, 2021.
(60) Provisional application No. 63/279,617, filed on Nov. 15, 2021.
(60) Provisional application No. 63/279,644, filed on Nov. 15, 2021.
(60) Provisional application No. 63/279,631, filed on Nov. 15, 2021.
(60) Provisional application No. 63/300,577, filed on Jan. 18, 2022.
(60) Provisional application No. 63/314,191, filed on Feb. 25, 2022.
(60) Provisional application No. 63/314,171, filed on Feb. 25, 2022.

(51) Int. Cl. C12N 5/071; C12N 5/00

(52) U.S. Cl. CPC C12N 5/0697; C12N 5/0018

(57) ABSTRACT

The present disclosure generally relates, in certain aspects, to cultivated meat and other cultivated animal-derived products. In some embodiments, the whole blood of a non-human animal is separated into various components (e.g., concentrated red blood cells, blood plasma, etc.). Some embodiments are generally directed toward incorporating coloring or “redness” into a product. For example, colorants may be added to a cultivated meat product to improve its color. The colorant may comprise a lysate of non-human red blood cells, e.g., containing hemoglobin. In some embodiments, the lysate may be obtained from blood withdrawn from living animal donors. Other embodiments yet are generally directed toward the composition and method of use of the lysate, cultivated meat products, kits involving these, or the like.

### RELATED APPLICATIONS

This application claims the benefit of U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Serial No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.” Each of these is incorporated herein by reference in its entirety.

### FIELD

The present disclosure generally relates to cultivated meat and other cultivated animal-derived products.

### BACKGROUND

Cultivated meat, or cell-based meat, is meat that is produced using in vitro cell culture or bioreactors, instead of being harvested from live animals. In many cases, the meat that is produced may include muscle cells and fat cells. Such meat may include, for example, chicken, beef, pork, or fish. Such technologies have the potential to revolutionize agriculture, for example, by decreasing the amount of land necessary to produce meat, avoiding unethical farming of animals, or increasing the available food supply. However, cultivated meats are difficult and expensive to manufacture. In addition, they often lack hemoglobin and myoglobin, and hence, are generally colorless or opaque, and do not have the “red” appearance and taste associate to red meat, and thus improvements are needed.

### SUMMARY

The present disclosure generally relates to the production of cultivated meat and other applications. The subject matter of the present disclosure involves, in some cases, interrelated products, alternative solutions to a particular problem, and/or a plurality of different uses of one or more systems and/or articles.

In accordance with certain embodiments, to produce cultivated meat product, various elements of the cultivated meat product can be obtained from blood, for example, obtained by separating the blood into various components (e.g., concentrated red blood cells, blood plasma, etc.). Some embodiments are generally directed toward incorporating coloring or “redness” into the cultivated meat. Cultivated meat products, for example, often may lack myoglobin, the iron-rich protein that gives meat its red color. Instead, such products may appear opaque or white. The lack of myoglobin may also affect the taste of the cultivated meat product. As such, one set of embodiments is generally directed to adding colorants to the cultivated meat product. In some cases, a colorant may comprise a lysate of non-human red blood cells, e.g., containing hemoglobin. In some embodiments, the lysate may be obtained from blood withdrawn from living animal donors. In certain cases, the animals are not intended to be slaughtered for meat consumption.

In some embodiments, the cultivated meat may be grown in a bioreactor comprising a cell culture media that is, at least partially, comprised of blood products. The blood products may be harvested from a human or non-human and may comprise components such as platelet rich plasma (PRP), platelet poor plasma (referred to as plasma), a platelet concentrate, a lysate of red blood cells, a platelet lysate (PL), growth factors, proteins, cytokines, or the like. In another embodiment, the non-human blood plasma may be used as a nutrient source in a bioreactor. In certain embodiments, the serum is fetal bovine serum. In some cases, the blood may be obtained from commercial vendors. However, in some embodiments, the non-human blood plasma may be obtained from living animal donors.

In addition, certain embodiments are directed to the assembly of the cultivated meat product. For instance, in some embodiments, a cultivated meat product is formed by mixing a lysate of non-human red blood cells with non-human animal cells (e.g., as a muscle replica) and/or a fat replica (e.g., comprising a fat emulsion and non-human blood plasma).

One aspect is generally directed to a cultivated meat product. In one set of embodiments, the cultivated meat product comprises a heme, a fat replica, and non-human animal cells.

In another set of embodiments, the cultivated meat product comprises a tissue mass of at least 10 g, comprising at least 0.1 wt % non-human red blood cell lysate, and microcarriers comprising fibrin.

Another aspect is generally directed to a method. In one set of embodiments, the method comprises lysing non-human red blood cells to produce a cell lysate, and mixing the lysate of non-human red blood cells and non-human cells to produce a tissue mass of at least 10 g.

The method, in another set of embodiments, comprises withdrawing a first blood draw from a non-human animal, causing at least a portion of the first blood draw to be formed into a first cultivated meat product, after at least 4 weeks after withdrawing the first blood draw, withdrawing a second blood draw from the non-human animal in quantity that is not harmful to animal, and causing at least a portion of the second blood draw to be formed into a second cultivated meat product.

In another aspect, the present disclosure encompasses methods of making one or more of the embodiments described herein, for example, cultivated meat. In still another aspect, the present disclosure encompasses methods of using one or more of the embodiments described herein, for example, cultivated meat.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates a process of separating whole blood to obtain packed non-human red blood cells and blood plasma, in accordance with one embodiment;

FIG. 2 illustrates the use of osmotic imbalance to lyse red blood cells to prepare a solution of lysate of non-human red blood cells, in accordance with another embodiment;

FIG. 3 illustrates the use of a sonicator (or other acoustic energy) to lyse red blood cells to prepare a solution of lysate of non-human red blood cells, in accordance with some embodiments;

FIG. 4 illustrates the use of freeze-thawing to lyse red blood cells to prepare a solution of lysate of non-human red blood cells, according to still another embodiment;

FIG. 5A illustrates a side-by-side visual comparison of the redness of cultivated meat products versus native group beef purchased at a grocery store, in accordance with some embodiments; and

FIG. 5B illustrates a side-by-side comparative color profile analyses of cultivated meat products versus native ground beef purchased at a grocery store, in accordance with some embodiments.

### DETAILED DESCRIPTION

The present disclosure generally relates, in certain aspects, to cultivated meat and other cultivated animal-derived products. In some embodiments, the whole blood of a non-human animal is separated into various components (e.g., concentrated red blood cells, blood plasma, etc.). Some embodiments are generally directed toward incorporating coloring or “redness” into a product. For example, colorants may be added to a cultivated meat product to improve its color. The colorant may comprise a lysate of non-human red blood cells, e.g., containing hemoglobin. In some embodiments, the lysate may be obtained from blood withdrawn from living animal donors. Other embodiments yet are generally directed toward the composition and method of use of the lysate, cultivated meat products, kits involving these, or the like.

One aspect of the present disclosure is generally directed toward providing a cultivated meat product (or other cultivated animal-derived product) having a relatively natural meat color. Cultivated meat products produced in vitro often do not contain sufficient myoglobin, the heme-containing protein that gives natural meat its color (usually a red color). For instance, natural meat appears red when myoglobin binds oxygen and changes the oxidation state of the iron group within the heme moiety (from Fe²⁺ to Fe³⁺).

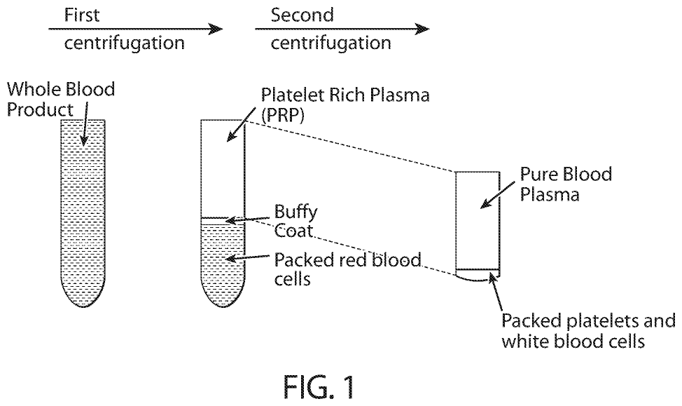

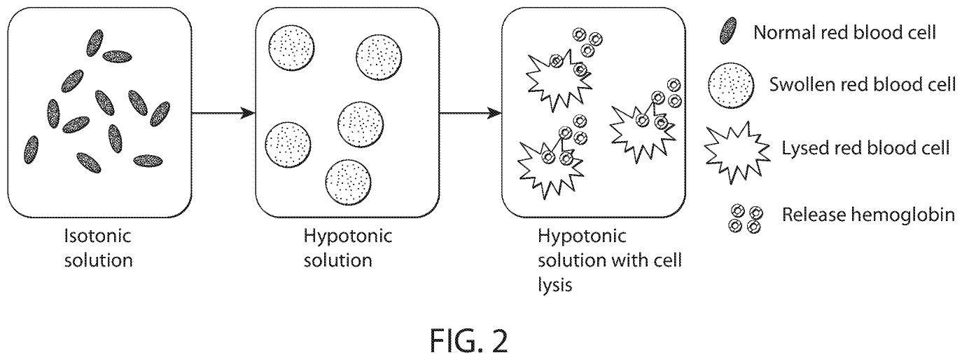

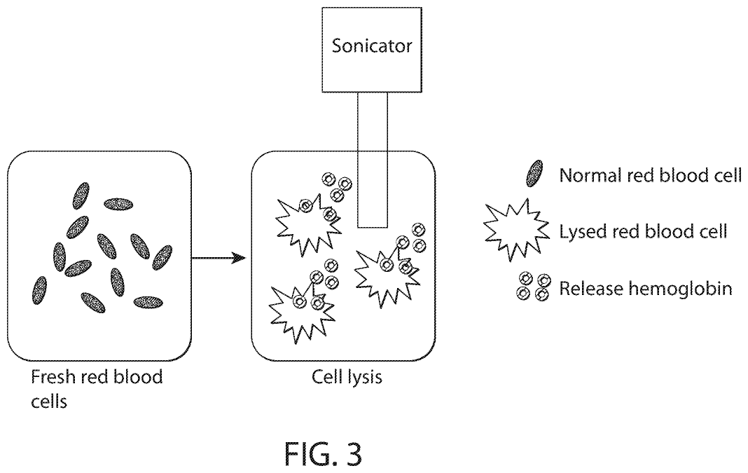

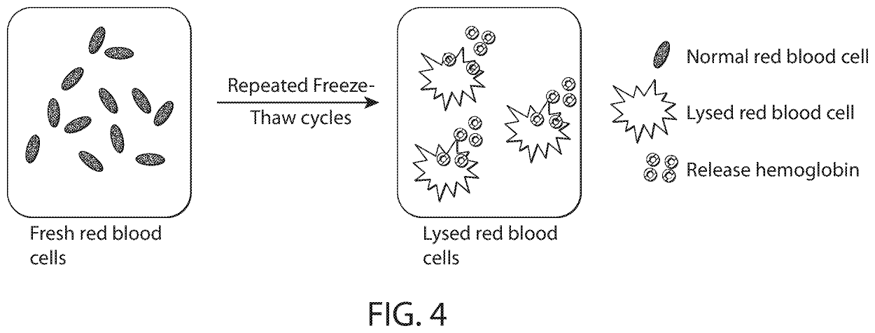

As such, one set of embodiments is directed toward introducing heme into a product, such as a cultivated meat product. In one embodiment, the heme may be obtained from non-human red blood cells. For example, red blood cells may be lysed, e.g., by exposing the cells to hypoosmotic or distilled water to form a lysate of non-human red blood cells (FIGS. 1-4). Red blood cells contain hemoglobin, a structurally similar protein to myoglobin. Hemoglobin also contains a heme moiety. Lysing the red blood cells may release hemoglobin, e.g., into solution. Furthermore, in addition to these, other methods of lysing red blood cells can be used, including those discussed in more detail herein.

Some embodiments are also directed toward repeated blood collection from non-human animals, for example, to obtain blood, blood serum, blood plasma, red blood cells, or other blood products, etc. For instance, in some embodiments, blood may be withdrawn from the animal at spaced intervals, so as to allow the animal time to recover and produce new blood. For instance, blood may be withdrawn from the animal every 2 weeks, every 4 weeks, every 6 weeks, every 2 months, or the like. The blood draws may each be processed, for example, as discussed herein. For example, the blood may be used to form a cultivated meat product. In this way, blood can be obtained in certain embodiments in a sustainable and cost-effective manner, without killing the animal. This usage may result, in certain embodiments, in the reduction in carbon emissions, water use, land use, etc.

Certain structures and methods described herein can be useful, for example, by providing meat and other animal derived products for human consumption. Certain embodiments of structures and methods described herein may offer certain advantages as compared to existing agriculture-based methods of meat production, for example, by significantly reducing the number of animals bred for slaughter, thus decreasing the number of foodborne illnesses, diet related diseases, and the incidence of antibiotic resistance and infectious disease (e.g., zoonotic diseases such as Nipah virus and influenza A). In some cases, reducing the number of livestock worldwide may also have an effect on the environmental risks associated with agricultural farming due to, for example, ammonia emissions which contribute significantly to acid rain and acidification of ecosystems. In addition, in some instances, livestock, such as pigs and cows are a major agricultural source of greenhouse gases worldwide. In some embodiments, the structures and methods described herein may allow meat and other animal-derived products to be produced or cultivated in vitro, e.g., using blood and tissue donations obtained from living livestock donors (e.g., not intended for slaughter for human consumption). As a non-limiting example, certain embodiments as described herein are generally directed to a product comprising a muscle replica, a fat replica, and a lysate of red blood cell.

The above discussion are non-limiting examples of certain aspects generally directed to cell lysates or methods of withdrawing blood from an animal, e.g., for various uses. However, other embodiments are also possible besides those discussed above.

For example, certain aspects are generally directed to cultivated animal-derived products, such as cultivated meat, or other products. These may be produced, for example, using cells taken from an animal, but then the cells are cultured in vitro, e.g., using bioreactors, flasks, petri dishes, microwell plates, or other cell culture systems. Many cell culture systems will be known to those of ordinary skill in the art. This is in stark contrast to traditional techniques of sacrificing animals and harvesting their meat or other organs (e.g., skin, internal organs, etc.) for food or other uses. Although the original cells seeded to form the product may have originated or otherwise have originally been derived from a living animal, the bulk of the cells forming the actual product were grown or cultured in an in vitro setting, rather than naturally as part a living animal.

A variety of products may be formed from cells cultured in vitro. For instance, in certain embodiments, the products may form “cultivated meat,” or meat that is intended to be eaten, for example, by humans. It will be appreciated that, because it is to be eaten, such products will often be formed of edible or digestible materials, e.g., materials that can be digested, or degraded to form generally nontoxic materials within the digestive system. For instance, the cultivated meat may contain animal-derived cells (e.g., derived from a chicken, a cow, a pig, a sheep, a goat, a deer, a fish, a duck, a turkey, a shrimp, or other animals that are commonly recognized for widespread human consumption), such as muscle cells, fat cells, or the like. The cells may be wild-type or naturally-occurring cells (e.g., harvested from an animal), although in some embodiments, the cells may include genetically engineered cells, e.g., engineered in a way to increase proliferation. In addition, in some embodiments, the cultivated meat product may contain other edible materials, such as plant-originated materials. Non-limiting examples of edible materials include proteins, carbohydrates, sugars, saccharides, plant-based fats, etc., as well as polymers formed from these (for example, polylactic acid, polyglycolic acid, cellulose, etc.). In some cases, the edible materials may be digested to form nutrients, e.g., such as amino acids, sugars, etc. that have nutritional value, for example, when taken up into the body. However, in some cases, the edible materials cannot be digested, and/or can be digested to form non-nutrients that cannot be absorbed as nutrients, but can be passed through the digestive system without detrimental effects.

In addition, it should be understood that the invention is not limited to only cultivated meat products. In some cases, products such as those described herein may be cultivated from animal-derived cells, but the product is not necessarily one that is intended to be eaten. For instance, cells from an animal may be cultured to form various organs that can be harvested, such as skin, hair, fur, or the like. Thus, as a non-limiting example, leather, cultivated fur, etc. can be formed by growing cells in culture, for example as discussed herein, without the traditional method of sacrificing animals to harvest their skin or other organs.

In certain embodiments, the cultivated meat products may be grown on microcarriers or other types of scaffolds, which may comprise fibrin in some embodiments. For example, cells derived from an animal may be seeded onto microcarriers or scaffolds, and grown in vitro, e.g., in a bioreactor or other cell culture systems such as are described herein, to produce a cultivated meat product (or other cultivated animal-derived product). In some cases, the product thus formed can be used without additional processing. As a non-limiting example, a cultivated meat product may be grown by seeding myoblasts on microcarriers or scaffolds, then grown within a bioreactor to form a muscle replica or a cultivated meat product, etc. The cultivated meat product may not require subsequent separation or processing steps to convert the cultured cells into a product ready to be cooked or otherwise be used, e.g., as meat. However, it should be understood that in other embodiments, additional steps may be used to convert the muscle replica grown within the bioreactor into a cultivated meat product, or other cultivated animal-derived product.

One set of embodiments is generally directed to the production and use of heme, which may be added to a product, such as a cultivated meat product, or other cultivated animal-derived products such as those discussed herein. The heme may arise from any suitable source. For instance, in certain embodiments, heme may be obtained from a lysate of non-human red blood cells. Such a lysate may be produced, for example, by lysing red blood cells, using any of a variety of lysing techniques. For example, the non-human red blood may arise from a chicken, a cow, a pig, a sheep, a goat, a deer, a fish, a duck, a turkey, a shrimp, or other animals.

For example, in one set of embodiments, cells may be lysed by exposing them to hypoosmotic water, such as distilled water. Cells typically have an osmolarity of about 300 mOsm; hypoosmotic solutions with osmolarities less than this (including distilled water, having an osmolarity of approximately zero) may cause water to enter the cells, ultimately causing them to burst or lyse. Accordingly, in certain embodiments, red blood cells are exposed to a hypoosmotic solution to cause the cells to lyse. In some embodiments the cells may be exposed to a volume of hypoosmotic water that is at least sufficient to lyse the red blood cells. For example, the volume may be at least 5%, at least 10%, at least 20%, at least 30%, at least 40%, or at least 50% of the volume of the solution containing the cells. These precents are percent by volume.

In addition, other methods of lysing red blood cells can be used in other embodiments. As one non-limiting example, acoustic energy may be used to lyse the cells. For example, the cells may be exposed to a sonicator or an ultrasonic bath to cause the cells to lyse. As yet another non-limiting example, the cells may be exposed to one or more freeze-thaw cycles, e.g., where the temperature is lowered to below the freezing point of the solution, thereby causing ice to form, killing at least some of the cells.

In certain embodiments, the cells may be lysed such that at least 50% of the cells have been lysed or ruptured, and in some embodiments, such that at least 60%, at least 70%, at least 80%, at least 85%, at least 90%, at least 95%, or at least 99% of the cells have been lysed or ruptured. One or more than one lysing technique may be used in various embodiments.

In addition, it should be understood that red blood cells are not the only source of heme. In another embodiment, for example, the heme may be obtained from plant globin, for example, by extracting heme proteins from the root nodules of various plants. In some embodiments the plant globin is leghemoglobin. Non-limiting examples of plants containing globins include beans, soybeans, peas, chickpeas, peanuts, lentils, lupins, mesquites, carob, tamarind, alfalfa, clover, etc. In another embodiment, heme iron may be obtained from organisms such as oysters, clams, mussels, chicken livers, organ meats, sardines, segmented worms, etc. The heme, in accordance with another embodiment, may also be produced using a genetically modified organism (GMO). In some embodiments the GMO is a yeast.

In addition, some embodiments are generally directed to forming a cultivated meat product, or other product such as is disclosed herein. For instance, in one set of embodiments, a product is formed using a cell lysate to produce a tissue mass. The cell lysate may be prepared as discussed herein. In some cases, the tissue mass of the mixture may be relatively large. For example, a cultivated meat product may comprise mixing a lysate of non-human red blood cells to product a tissue mass of at least 1 g, of at least 5 g, of at least 10 g. In some embodiments, the lysate may be mixed with non-human animal cells (e.g., as a muscle replica), and/or a fat emulsion and non-human blood plasma (e.g., as a fat replica), etc., to produce a cultivated meat product, or another cultivated animal-derived product. Examples of muscle replicas include those disclosed in a patent application entitled “Constructs Comprising Fibrin or Other Blood Products for Meat Cultivation and Other Applications,” U.S. patent application Ser. No. 63/279,617; examples of fat replicas include those disclosed in a patent application entitled “Systems and Methods of Producing Fat Tissue for Cell-Based Meat Products,” U.S. Pat. Apl. Ser. No. 63/279,642. Each of these was filed on Nov. 15, 2021, and is incorporated herein by reference in its entirety.

In some embodiments, at least 1 wt %, at least 3 wt %, at least 5 wt %, at least 10 wt %, at least 15 wt %, at least 20 wt %, at least 25%, at least 30 wt %, at least 40 wt %, at least 50 wt %, etc. of the product comprises the lysate of non-human red blood cells. In addition, in certain embodiments, no more than 50 wt %, no more than 40 wt %, no more than 30 wt %, no more than 25 wt %, no more than 20 wt %, no more than 15 wt %, no more than 10 wt %, no more than 5 wt %, no more than 3 wt %, or no more than 1 wt % of the product comprises the lysate of non-human red blood cells. Combinations of any of these are also possible in certain cases; for example, the product may have between 5 wt % and 10 wt %, between 10 wt % and 25 wt %, between 1 wt % and 20 wt %, etc. of a lysate.

In some instances, a product such as a cultivated meat product further comprises binding agents that hold the various components together. Exemplary embodiments include transglutaminase, non-human plasma, fibrinogen, soy isolate, a soy concentrate, a soy milk, an egg, a soy flour, a wheat gluten isolate, or a pea isolate.

However, it should be understood that the present disclosure is not limited to only the production of heme from non-human blood. Other embodiments are generally directed to other uses for non-human blood, e.g., to produce a cultivated meat product, or other products such as are discussed herein. For instance, certain embodiments are generally directed to non-human blood plasma, which may be prepared from non-human blood.

For example, in one embodiment, non-human blood plasma may be used as a source of fibrinogen to form fibrin hydrogels, which may be used to form fibrin microcarriers, for example by milling or other techniques. In some embodiments, fibrin microcarriers may be seeded with non-human cells, e.g., myoblasts cells, adipose cells, etc. In some embodiments, a fat replica may be formed using the non-human blood plasma to create a clot or a fibrin hydrogel, e.g., by exposure to thrombin, calcium, etc.

As another example, in some embodiments, non-human blood plasma may be used for the cultivation of cells in bioreactors. Cells typically require a variety of compounds such as soluble proteins, growth factors, cytokines, etc. for various activities, such as adhesion, proliferation, differentiation into different cell types, etc. In some embodiments, for example, non-human blood plasma may be applied to cells, e.g., in a bioreactor or other reactors such as those described herein.

As mentioned, some embodiments are directed toward repeated blood collection from non-human animals, for example, to obtain blood, blood serum, blood plasma, red blood cells, or other blood products, etc. For example, repeated blood collection from non-human animals may be used to obtain non-human blood plasma, lysate of non-human red blood cells, or the like.

In some cases, blood may be withdrawn from a donor animal at spaced intervals, which may be regular or irregular. Between blood draws, the animal can recover and produce new blood. Any suitable interval may be used. For example, the blood may be withdrawn from the animal every 2 weeks, every 4 weeks, every 6 weeks, every 2 months, or the like. As an example, in some embodiments, a blood draw may be withdrawn from an animal, and after a suitable interval, an additional blood draw may be withdrawn from the animal. This cycle can also be repeated any suitable number of times. The blood draws may each be processed, for example, as discussed herein. For example, the blood may be used to form a cultivated meat product.

For example, in one set of embodiments, one or more blood draws may be withdrawn or taken from a non-human animal. Exemplary embodiments of non-human animals include chicken, cow, pig, mutton, goat, deer, fish, duck, turkey, shrimp, or other animals that are commonly recognized for widespread human consumption. The blood may be processed in some cases to isolate the non-human red blood cells, e.g., using centrifugation, apheresis, or other suitable techniques. In another set of embodiments, the non-human red blood cells may be lysed, e.g., as discussed herein. For example, the lysate may be made into a first cell-based meat product (e.g., using the lysate of non-human red blood as colorant). In some embodiments, the non-human red blood cells are lysed within 24 hours of withdrawal from a non-human living donor to produce the lysate. In another set of embodiments, non-human blood plasma may be used to form microcarriers, e.g., microcarriers comprising fibrin. In yet another set of embodiments, the non-human blood plasma may be used as a cell culture additive, e.g., to culture cells in a bioreactor.

In addition, the animal may also be subjected to second, third, etc. blood draws, e.g., at spaced intervals such as discussed herein. The blood withdrawn in each draw may be treated in the same way, or in different ways, depending on the application. Notably, however, the animal remains alive between blood draws, and can recover and produce new blood. In this way, although blood production from the animal occurs, the animal is not slaughtered in order for blood production from the animal to continue.

In some embodiments, it is believed that withdrawing blood from living animal donors, as opposed to farming them for slaughter, may translate into substantial environmental improvements, such as reductions in carbon emissions, water, land, etc.

In some embodiments, a cultivated meat product as discussed herein comprises a fat replica, such as discussed in a patent application entitled, “Systems and Methods of Producing Fat Tissue for Cell-Based Meat Products” filed on Nov. 15, 2021, U.S. Pat. Apl. Ser. No. 63/279,642, incorporated herein by reference in its entirety. In one embodiment, this native structure is replicated by seeding non-human fat cells, such as adipose cells or adipose progenitor cells, onto microcarriers, or other scaffolding material, and culturing them in a bioreactor, or other in vitro cell culture system, to form the fat replica. In some embodiments, the cells are grown in serum to support cell proliferation and then induced to differentiate.

In some embodiments, the fat may be present within the fat replica as an emulsion. For example, the fat may be contained within fat particles in the fat replica. In some cases, the fat may be dispersed (for example, homogenously) within the emulsion. In one set of embodiments, for example, fat particles may be formed by mixing fat with non-human blood plasma, which are suspended as an emulsion. The non-human blood plasma may come from any suitable source. The plasma may arise from the same, or a different type of animal than the fat or fat cells that may be present.

In addition, in certain embodiments, a surfactant may be used to disperse the fat and/or stabilize the emulsion. Non-limiting examples of surfactants include phospholipids, monoglyercols, diglycerols, propylene glycol monoesters, lactylate esters, polyglycerol esters, sorbitan esters, ethoxylated esters, succinate esters, fruit acid esters, acetylated monoglycerols, acetylated diglycerols, phosphate monoglycerols, phosphate diglycerols, sucrose esters, etc. For example, a surfactant may be mixed with animal cells and/or non-human blood plasma to form an emulsion.

In addition, in some embodiments, a cultivated meat product as discussed herein comprises a muscle replica, such as discussed in a patent application entitled, “Constructs Comprising Fibrin or Other Blood Products for Meat Cultivation and Other Applications” filed on Nov. 15, 2021, U.S. Pat. Apl. Ser. No. 63/279,617, incorporated herein by reference in its entirety. For example, the muscle replica may comprise non-human muscle cells seeded on microcarriers. In some embodiments, the microcarriers comprise fibrin. In certain embodiments, microcarriers such as those discussed herein may be treated to facilitate binding of cells, such as myoblasts and/or adipose cells. For example, the microcarriers may be exposed to non-human serum, which may include growth factors that bind to the microcarriers. The growth factors may, for example promote cell adhesion, proliferation, and/or migration of cells into the microcarriers. In addition, in some cases, the microcarriers may have structures, such as grooves, that may allow the cells such as myoblasts to become aligned in a specific direction, although this is not a requirement. Such structures are described in U.S. Ser. No. 63/159,403, filed Mar. 10, 2021, entitled “Constructs for Meat Cultivation and Other Applications,” by Khademhosseini, et al., incorporated herein by reference.

In some embodiments, the microcarriers or scaffolds may comprise any material that forms an edible hydrogel, such as fibrin. For example, in one embodiment, a microcarrier may be formed from a non-human blood plasma which contains plasma-rich fibrinogen that can be crosslinked or otherwise processed to form a fibrin hydrogel. Such crosslinking can be achieved by exposure to thrombin, calcium, or other conditions such as those described herein. In some embodiments, fibrin hydrogels are formed using non-human blood plasma containing fibrinogen, e.g., at least 10 wt %, or more in some cases.

In certain embodiments, non-human cells such as muscle cells, adipose cells, etc. may be seeded on the microcarriers or other scaffolds, and grown in a bioreactor or other in vitro cell culture system. In one set of embodiments, a scaffold may have a largest or maximum internal dimension of less than 100 mm, less than 80 mm, less than 70 mm, less than 60 mm, less than 50 mm, less than 40 mm, less than 30 mm, less than 20 mm, less than 10 mm, less than 5 mm, less than 3 mm, less than 2 mm, or less than 1 mm. In addition, in some cases, the microcarriers may have a maximum internal dimension that is at least 1 mm, at least 2 mm, at least 3 mm, at least 5 mm, at least 10 mm, at least 20 mm, at least 30 mm, at least 40 mm, at least 50 mm, at least 60 mm, at least 70 mm, at least 80 mm, at least 90 mm, at least 100 mm, etc. Combinations of any of these dimensions are also possible in some embodiments.

The scaffold may comprise any suitable material. For example, in one set of embodiments, the scaffold may comprise fibrin, or another edible material. This may be useful for applications such as cultivated meat, where the cultivated animal-derived product will be eaten, e.g., by humans or other animals. In some embodiments, the microcarriers may comprise a hydrogel, e.g., a fibrin hydrogel, or other hydrogels such as those described herein.

In some cases, at least 50 wt %, at least 60 wt %, at least 70 wt %, at least 80 wt %, at least 90 wt %, or substantially all of a scaffold is formed from fibrin, and/or another edible material. The fibrin may arise from any suitable source. For example, the fibrin may arise from a non-human animal, such as a non-human mammal. Non-limiting examples include cows, pigs, sheep, goats, or the like. In some cases, the fibrin may arise from the blood of such an animal. For instance, in some embodiments, the fibrin may be prepared by acquiring blood or blood plasma from an animal, and processing it to produce fibrin. For example, in one set of embodiments, the blood is exposed to a protease inhibitor such as thrombin, which may cause fibrinogen to clot to form fibrin. The fibrin may be harvested, and used as discussed herein, e.g., to produce scaffolds such as microcarriers. In addition, in some cases, fibrin may be obtained from fibrinogen, which may be bought commercially, obtained from blood plasma, or the like.

The fibrin may be processed to form a scaffold. In one set of embodiments, the scaffold may take the form of one or more microcarriers. The microcarriers may have any shape or size. In some cases, more than one type of microcarrier may be present, e.g., some of which may have various materials, shapes, sizes, etc., such as are described herein. For example, in some embodiments, at least some of the microcarriers may be substantially spherical or exhibit spherical symmetry, although in other embodiments, at least some of the microcarriers may be non-spherically symmetric (for example, triangular) or may be anisotropic. In addition, in certain cases, at least some of the microcarriers may have a plurality of grooves, e.g., as discussed herein.

Fibrin itself may be edible. The microcarrier or scaffold may comprise, in addition to or instead of fibrin, other edible materials in certain embodiments. In addition, it should be understood that the scaffold is not limited to only edible or degradable materials. In other embodiments, the scaffold may comprise materials, such as polymers, that are not necessarily edible and/or degradable. Non-limiting examples of such materials include natural polymers such as proteins (e.g., silk, collagen, gelatin, fibrinogen, elastin, keratin, actin, myosin, etc.), polysaccharides (e.g., cellulose, amylose, dextran, chitin, glycosaminoglycans), or the like. Other examples include polymers such as polylactic acid, polyglycolic acid, poly(lactic-co-glycolic acid), polyhydroxyalkanoates, polycaprolactones, etc., bioactive ceramics such as hydroxyapatite, tricalcium phosphate, silicates, phosphate glasses, glass-ceramic composites (such as apatite-wollastonite), etc., or the like.

Additionally, in some but not all embodiments, the scaffold may comprise a plant-originated material, such as a plant-originated protein. Such plant-originated materials may be harvested directly from a plant, be grown in vitro (e.g., in cell culture from a culture initially originating in a plant), be synthetically produced (e.g., without using a plant, e.g., chemically produced), etc. Examples of protein-originated material include, but are not limited to, cellulose or certain proteins, such as prolamin, zein, fibrin, gliadin, hordein, secalin, kafirin, avenin, gliadine, 2S albumin, globulin, glutelin, etc. The plant that material originates from may be any plant, including but not limited to food crop plants. Non-limiting examples of plants include, but are not limited to, wheat, barley, rye, corn, sorghum, oats, quinoa, hemp, potato, soy, etc. Additional examples of such materials include those described in U.S. Ser. No. 63/159,403, filed Mar. 10, 2021, entitled “Constructs for Meat Cultivation and Other Applications,” by Khademhosseini, et al., incorporated herein by reference in its entirety.

In some embodiments, the microcarriers may be formed into a cultivated meat product. For example, cells such as non-human animal cells may be grown on the microcarriers, e.g., as discussed herein. Non-limiting examples include adipose (fat) cells, muscle cells (e.g., myoblasts), or the like, and the cells may arise from the same or different species. In some cases, relatively large quantities of product may be prepared, e.g., by growing the cells in a bioreactor or other in vitro cell culture system, until at least a certain size or mass is reached. For example, the cells may be grown until they form a product that is, for example, at least 10 g, at least 25 g, at least 50 g, at least 100 g, at least 300 g, at least 1 kg, etc. Those of ordinary skill in the art will be aware of bioreactors and other cell culture systems.

As mentioned, in some aspects, a product such as a cultivated meat product may be produced within a bioreactor or other cell culture system. A wide variety of bioreactors can be used in various embodiments including, but not limited to, suspension bioreactors, continuous stirred-tank bioreactors, rocker bioreactors, airlift bioreactors, fixed bed bioreactors, bubble column bioreactors, fluidized bed bioreactors, packed bed bioreactors, or the like.

In some cases, cells may be seeded on microcarriers or other scaffolds, then introduced into the bioreactor or other cell culture system. Those of ordinary skill in the art will be familiar with techniques for seeding cells on a scaffold. For instance, the scaffold may be exposed to a suspension containing animal-derived cells, which are allowed to settle from the suspension onto the scaffold. In some cases, one or more than one type of cell may be present in suspension and allowed to settle.

In some cases, a product can be formed within the bioreactor without additional processing, for example, without separating the cells or tissues grown within the bioreactor. However, in other cases, some separation and/or processing of the cells may be used. As a non-limiting example, myotubes may be grown within a bioreactor or other cell culture system such as those described herein to produce a muscle replica. In some embodiments, such muscle replicas may be processed, e.g., by adding a fat replica to produce a cultivated meat product having any desired ratio of muscle to fat in it. For instance, the ratio of muscle to fat may be at least 95:1, at least 90:1, at least 70:1, at least 50:1, at least 30:1, at least 20:1, at least 10:1, at least 5:1, at least 1:1, etc. by weight. One non-limiting example of a fat replica is a fat emulsion and non-human blood plasma, e.g., as discussed in a patent application entitled “Systems and Methods of Producing Fat Tissue for Cell-Based Meat Products,” filed on Nov. 15, 2021, U.S. Pat. Apl. Ser. No. 63/279,642, incorporated herein by reference in its entirety.

A variety of techniques may be used to grow cells within the bioreactor or other cell culture system. For instance, the cells may be grown at body temperature (e.g., about 38.5° C. for cow cells, about 41° C. for chicken cells, about 39-40° C. for pig cells, about 40-42° C. for duck cells, etc.). In some embodiments, during cultivation, the cells may have a shear stress applied to them of at least 0.005 newton/meters squared, of at least 0.1 newton/meters squared, of at least 0.2 newton/meters squared, of at least 0.3 newton/meters squared, of at least 0.4 newton/meters squared, of at least 0.5 newton/meters squared, of at least 0.6 newton/meters squared, of at least 0.7 newton/meters squared, of at least 0.8 newton/meters squared, etc.

In some embodiments, cells within the bioreactor or other cell culture system may be induced to differentiate, e.g., by adding suitable factors and/or altering the cell culture conditions therein. As a non-limiting example, myoblasts may be grown in serum, while removing or reducing the serum from the myoblasts may cause the myoblasts to differentiate to from myotubes. For instance, in one set of embodiments, the serum may be reduced from 10% to 2% to induce differentiation of myoblasts. Those of ordinary skill in the art will be aware of methods and systems to induce differentiation in cells.

In one embodiment, the cell-based meat product may be grown in a bioreactor, or other in vitro cell culture system, comprising a cell growth medium. In one embodiment, the cell growth medium comprises an animal derived product, for example, platelet rich plasma (PRP), platelet poor plasma, platelet lysate (PL), platelet concentrate, a lysate of red blood cells, optionally comprising other nutrients, or the like. The cell growth medium may be used for the production of cell-based meat and/or to enhance the proliferation of primary cells, stem cells such as myoblasts, fibroblasts, adipocyte, vascular, osteoblasts, tenocyte, neural cells, etc. These cells may be isolated from human or non-human animals, grown in vitro, etc. These may include but are not limited to humans, cows, sheep, swine, horses, goats, camels, whales, fishes, crabs, shrimp and the like. In some embodiments, the blood products may be obtained from the blood of animals destined to slaughtered for food.

In one embodiment, the platelet rich plasma (PRP) may be derived from whole blood from which red blood cells and white blood cells have been removed, such as by centrifugation, filtration, or other techniques known to those of ordinary skill in the art. Platelet rich plasma (PRP) may be generally categorized based on its leukocyte and fibrin content as (1) leukocyte-rich PRP (L-PRP), (2) leukocyte reduced PRP (P-PRP); (3) leukocyte reduced/pure PRP, or (4) leukocyte platelet-rich fibrin/pure platelet-rich fibrin (L-PRF). The platelet-rich plasma may be a blood derived composition having an increased concentration of platelets, compared to normal blood. For example, the PRP may have at least double, at least five times, or at least ten times or more the normal concentration of platelets in blood. In addition, in accordance with another embodiment, the platelet rich plasma may contain a variety of endogenous growth factors, such as transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, etc.

In one embodiment, the platelet concentrate (PC) may be derived from the platelet rich plasma (PRP), for example, by centrifugation. In some embodiments, the concentration may be at least 10³ platelets/mL, at least 10⁴ platelets/mL, at least 10⁵ platelets/mL, at least 10⁶ platelets/mL, at least 10⁷ platelets/mL, at least 10⁸ platelets/mL, at least 10⁹ platelets/mL, at least 10¹⁰ platelets/mL, etc. In some embodiments, donated platelet concentrates may be stored at 4° C. prior to use for at least 1 day, at least 2 days, at least 3 days, at least 4 days, at least 5 days, at least 6 days, or at least 7 days after donation. In another embodiment, expired human platelet concentrate may be obtained from blood banks, hospitals, and other institutions that routinely collect and store platelet rich plasma, and used as an additive in the cell growth medium.

In one embodiment, the platelet concentrate may impart the cell growth medium with antimicrobial properties. Platelets have certain properties similar to immune cells, and can in some cases induce potent anti-inflammatory responses when exposed to a number of chemical and biological triggers, for example, liposaccharide protein (LPS). In some embodiments, the platelet concentrate may be added to the cell culture medium and stimulated by treatment with platelet activating reagents, for example, calcium, thrombin, citrate, EDTA, plasminogen, and other platelet activating reagents known to those skilled in the art, e.g., to release antimicrobial molecules that may neutralize common bacterial, fungal, or viral food pathogens. In some embodiments, an acellular antimicrobial cell growth medium may be prepared by first culturing the platelet concentrate in the cell culture medium, stimulating them to release their antimicrobial payload, and then separating the antimicrobial cell growth medium from the platelet concentrate.

In one embodiment, the platelet concentrate may be lysed, for example by freeze-thawing or physical shearing (e.g. sonication or homogenization, etc.), to yield a platelet lysate (PL) comprising a plurality of cytokines and growth factors (e.g. transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, etc.) that in some embodiments may enhance cell proliferation, for example, of myoblasts and adipocytes. In some embodiments, the platelet lysate comprises human platelets, and/or non-human platelets. For example, in one embodiment, the platelet rich plasma may include bovine platelet rich plasma.

In one embodiment, the cell growth medium comprises a combination of a platelet lysate (PL) and a platelet rich plasma (PRP). In one embodiment, the PL/PRP comprise at least 2 to 20% w/v, at least 5-15% w/v, or at least 10% w/v of the cell culture growth medium. In another embodiment, the total platelet component in the cell growth medium is at least 2 to 5 mg/mL, at least 2 to 10 mg/mL, at least 2 to 20 mg/mL, or at least 9 to 11 mg/mL.

The cells may be grown within the bioreactor or other cell culture system for any suitable length of time, e.g., to produce a cultivated product. For example, the cells may be grown for at least 3 days, at least 5 days, at least 1 week, at least 2 weeks, at least 3 weeks, at least 4 weeks, etc.

The following are each incorporated herein by reference in their entireties: U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Serial No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.” The following examples are intended to illustrate certain embodiments of the present disclosure, but do not exemplify the full scope of the disclosure.

### Example 1

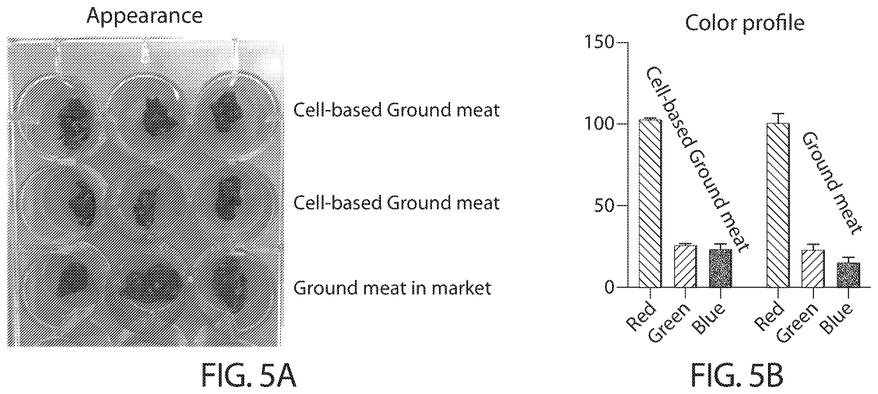

In this example, a cultivated meat product was produced by mixing bovine muscle cells with a lysate of non-human red blood cells. A muscle replica comprising bovine muscle cells was prepared, as discussed in a patent application entitled, “Constructs Comprising Fibrin or Other Blood Products for Meat Cultivation and Other Applications” filed on Nov. 15, 2021. Fresh whole blood was obtained from a living cow and the red blood cells were separated from the whole blood by centrifugation at 150 g for 25 minutes at 4° C. The isolated red blood cells were then lysed using distilled water to a final concentration of 20% v/v. Although non-lysed red blood cells could also be used as a colorant in other embodiments, in these examples, it was observed that higher concentrations of unlysed red blood cells may be needed to obtain the same color profile as lysed red blood cells (FIG. 5A, B).

In some embodiments, a red colorant was obtained by mixing a 3% (vol/vol) lysate of non-human red blood cells with 77% bovine muscle replica and 20% fat replica. These were mixed together to form a cultivated meat product resembling ground beef. In some embodiments, the cultivated meat product may be mixed with a binding agent and a crosslinking agent. The binding agent may be non-human plasma (20% vol/vol) and the crosslinking agent may be calcium chloride (0.1% w/v) or thrombin. In some embodiments, the cultivated meat product may be cut and pressed to remove excess water.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
lysing non-human red blood cells to produce a cell lysate; and
mixing the cell lysate and non-human cells to produce a tissue mass of at least 10 g.

2-59. (canceled)

60. The method of claim 1, wherein the cell lysate is present in the tissue mass at a concentration of at least 0.1%.

61. The method of claim 1, wherein the non-human red blood cells and the non-human cells arise from the same animal species.

62. The method of claim 1, wherein the non-human red blood cells and the non-human cells arise from different animal species.

63. The method of claim 1, comprising:
withdrawing a blood sample from a non-human animal; and
processing the non-human blood sample to produce the non-human red blood cells.

64. The method of claim 63, comprising processing the non-human blood sample using apheresis.

65. The method of claim 1, wherein the tissue mass comprises myoblasts.

66. The method of claim 1, wherein the tissue mass comprises microcarriers.

67. The method of claim 66, wherein the microcarriers comprise a hydrogel.

68. A method, comprising:
withdrawing a first blood draw from a non-human animal;
causing at least a portion of the first blood draw to be formed into a first cultivated meat product;
after at least 4 weeks after withdrawing the first blood draw, withdrawing a second blood draw from the non-human animal in quantity that is not harmful to animal; and
causing at least a portion of the second blood draw to be formed into a second cultivated meat product.

69. The method of claim 68, further comprising lysing at least 50% of blood cells the first blood draw.

70. The method of claim 69, further comprising:
after at least 4 weeks after withdrawing the second blood draw, withdrawing a third blood draw from the non-human animal; and
causing at least a portion of the third blood draw to be formed into a third cultivated meat product.

71. The method of claim 68, comprising withdrawing the second blood draw from the non-human animal after at least 6 weeks after withdrawing the first blood draw.

72. The method of claim 68, wherein the non-human animal is a cow.

73. A cultivated meat product, comprising:
a heme;
a fat replica; and
non-human animal cells.

74. The cultivated meat product of claim 73, wherein the heme comprises a non-human animal heme.

75. The cultivated meat product of claim 73, wherein the heme comprises a plant-based heme.

76. The cultivated meat product of claim 73, wherein the fat replica comprises a hydrogel.

77. The cultivated meat product of claim 73, wherein the fat replica comprises crosslinked non-human blood plasma.

78. The cultivated meat product of claim 73, wherein the non-human animal cells comprise muscle cells.
